(19) United States
(12) Patent Application Publication
(10) Pub. No.: US 2022/0409680 A1
(43) Pub. Date: Dec. 29, 2022

(54) NEW FORMULATION OF LACTOBACILLUS STRAINS FOR THE TREATMENT AND PREVENTION OF HELICOBACTER PYLORI COLONISATION IN THE UPPER AIRWAYS AND THE DIGESTIVE SYSTEM

(71) Applicant: BELANO MEDICAL AG, Henningsdorf (DE)

(72) Inventor: Alexander REIPRICH, Lychen (DE)

(21) Appl. No.: 17/777,075

(22) PCT Filed: Nov. 16, 2020
(86) PCT No.: PCT/DE2020/000282

§ 371 Date: May 16, 2022

(30) Foreign Application Priority Data
Nov. 14, 2019 (DE) 10 2019 007 892.6

(51) Int. Cl. A61K 35/747; A23G 4/12; A61K 35/745; A61K 9/68; A61K 9/00; A61P 31/04

(52) U.S. Cl. CPC A61K 35/747; A23G 4/123; A61K 35/745; A61K 9/0058; A61K 9/0073; A61P 31/04

(57) ABSTRACT

The invention relates to new compositions for the treatment and prevention of Helicobacter pylori colonisation of the oral cavity, the upper airways, the oesophagus, and the digestive system, wherein the formulation is preferably composed in the form of a chewing gum.

### FIELD OF THE INVENTION

The invention relates to new formulations for the treatment of Helicobacter pylori infections of the upper airways, the oesophagus, and the digestive system, wherein the formulation is preferably composed in the form of a chewing gum.

Prior art and background of the invention

It is known that Helicobacter pylori infections of the stomach may result in chronic inflammations and gastric ulcers. It is also known that H. pylori may also colonise outside the stomach, e.g., in dental plaques and gingival pockets, in aphthae, the palatine, and pharyngeal tonsils, and nasal polyps. H. pylori colonisation may be accompanied by digestion disorders, nausea, chronic mucosal ulcers, hives, anaemia, coronary circulatory disorders, insulin resistance, diabetes type 2, and gingival inflammations. Various preparations for the treatment of Helicobacter pylori infections are known, in particular antibiotics.

It is also known that certain Lactobacillus strains can be used against H. pylori infections. Some of these strains are mentioned in EP 1963483 B1, inter alia. Nevertheless, it was found out that in individual cases, despite initial treatment successes, a resurgence of the infection by reinfections occurs. There is, therefore, a need of improved formulations for known Lactobacillus strains, in order to fight H. pylori foci outside the gastro-intestinal tract and new infections by oronasal take-up of H. pylori in a better way.

It has now been found that, surprisingly, compositions, in which in principle known Lactobacillus strains, in particular, Lactobacillus reuteri, in particular Lactobacillus reuteri cells filed as DSM 17648, can be formulated in the form of a chewing gum, and that the application of such formulated chewing gums exceeds previous treatment successes outside the digestive system.

The term “Lactobacillus cells” in the meaning of the invention (in the following also lactic acid bacteria or lactobacilli) includes such microorganisms that require hydrocarbons, in particular glucose and lactose, for the lactic acid fermentation and in most cases the application of the Embden-Meyerhof biosynthesis pathway. The Lactobacillus cells can be used in living, viable, dead, or fragmented forms as cell wall constituents or in the form of isolated cell wall molecules. The Lactobacillus cells are taxonomically summarised in the family Lactobacteriaceae. They are gram-positive, non-spore-forming, and in general immobile. The Lactobacillus cells live anaerobically, and are, however, aerotolerant, although they do not include haemins (cytochrom, catalase) (Schleifer et al., System. Appl. Microb. 18:461-467 (1995) or Ludwig et al., System. Appl. Microb. 15:487-501 (1992).

According to the invention, in particular, such species are included that are suitable for homofermentative lactic acid fermentation or heterofermentative lactic acid fermentation.

Further, such Lactobacillus cells are preferably selected from the group Lactobacillus crispatus, Lactobacillus gasseri, Lactobacillus lectis, Lactobacillus helveticus, Lactobacillus acidophilus, Lactobacillus bulgaricus, Lactobacillus delbrueckii, Lactobacillus casei, Lactobacillus paracasei, Lactobacillus pentoses, Lactobacillus rhamnosus and Lactobacillus plantarum (all homofermentative), further Lactobacillus reuteri, Lactobacillus brevis, Lactobacillus fermentum, Lactobacillus viridescens as well as Bifidobacterium bifidum (all heterofermentative).

Lactobacillus reuteri is preferred according to the invention. Particularly preferred are Lactobacillus reuteri cells, as filed under DSM 17648.

The Lactobacillus cells can be used for the application according to the invention in living, viable, dead, or fragmented forms as cell wall constituents or in the form of isolated cell wall molecules.

Therein, the compositions may be in the form of pharmaceutical and/or dietary formulations and/or medical products, and/or cosmetic products.

Therefore, the invention relates to compositions comprising an effective dose of Lactobacillus cells in the form of a chewing gum for the treatment and prevention of H. pylori infections of the upper airways, oesophagus, and the digestive system.

In particular, the invention relates to compositions according to claim 1, comprising an effective dose, e.g., between 1×10³ and 1×10¹³ Lactobacillus reuteri cells in living, viable, dead, or fragmented forms. Further, cell wall constituents or isolated cell wall molecules respectively from 1×10³ to 1×10¹³ cells per application can also be used. The preferred dose includes 1×10⁹ to 1×10¹¹ Lactobacillus reuteri cells per application. The preferred application forms are viable or dead cells.

In particular, the invention relates to compositions according to claim 1, comprising an effective dose, e.g., between 1×10³ and 1×10¹³ Lactobacillus reuteri DSM 17648 cells in living, viable, dead, or fragmented forms. Further, cell wall constituents or isolated cell wall molecules respectively from 1×10³ to 1×10¹³ Lactobacillus reuteri DSM 17648 cells per application can also be used. The particularly preferred application forms are viable or dead Lactobacillus reuteri DSM 17648 cells.

The preferred dose includes 1×10⁹ to 1×10¹¹ Lactobacillus reuteri cells per application.

It seems to be crucial for the application that the compositions according to the invention remain over a longer period of time in the mouth/pharynx and continuously release the lactobacilli during this time. Alternatively, those compositions can be used for the chewing gum formulation, which usually have a longer residence time in the upper airways. Typically, the residence time in the mouth should be at least 30 seconds, preferably longer than 2 minutes, particularly preferably longer than 5 minutes, especially longer than 10 minutes.

In particular, e.g., the following administration forms can be used for the application:
1. Buccal tablets (also muco-adhesive buccal tablets) or orodispersible tablets,
2. oral active ingredient films,
3. gargle solutions or concentrates (incl. powders/tablets for the production),
4. mouth rinses or concentrates (incl. powders/tablets for the production),
   gingival cleaning solutions,
5. oromucosal preparations, such as drops, gel, liquids, pastes, solutions, sprays, and/or suspensions,
6. sublingual preparations, such as sprays, tablets, films,
7. lozenges or candies,
8. powders or granules for oral application,
9. preparations for nasal application, such as nose drops, liquid nose sprays, nose powders, semi-solid preparations for nasal application, nose rinses, or nose inhalers.

The chewing gums according to the invention can be produced, for instance, using a “gum base”, as offered by different companies, for instance, by Cafosa under the designation PWD 01, PWD 03, or PWD 04. Such compositions include, first, the actual gum mass, usually consisting of one or several elastomers, preferably selected from the group comprising polyisobutylene, isobutylene/isoprene copolymer, and vinyl acetate/vinyl laurate copolymer, polyvinyl acetate. Further, resins, preferably selected from the group comprising vegetable resin esters, resins and/or terpene resins produced by synthesis, hydrogenated or partially hydrogenated vegetable oils, and/or waxes selected from the group comprising vegetable waxes, crude oil-derived waxes and/or synthetic waxes may be included in the composition.

Furthermore, emulsifiers and technical means, more precisely, but not exclusively, glycerol monostearate, acetylated monoglycerides, lecithin, sugar esters, and triacetin, inert fillers such as maltodextrin, plant starch, silicon dioxide (E551), aerosil, magnesium stearate, and antioxidants are usually employed. Commonly, sweeteners, such as glucose, sucrose, or fructose are included. Preferably, however, the sweeteners are sugar-free and include, e.g., acesulfame, aspartame, cyclamate, saccharin, sucralose, thaumatin, neohesperidin, neotame, xylitol, mannitol, erythritol, isomalt, steviol glycoside, and/or sorbitol. For improving the taste, flavouring agents (e.g., strawberry flavour, vanilla flavour, raspberry flavour, banana flavour, chocolate flavour, mint flavour, thyme flavour, or sage flavour) and acidifiers such as, e.g., citric acid or lactic acid may be added.

To this basic chewing gum mixture are added the lactobacilli according to the invention (e.g., DSM 17648) and admixed with one another by intensive stirring. The addition of DSM 17648 can be made in the form of the product Pylopass (Novozymes A/S), in which DSM 17648 is commercially available. The basic chewing gum mixture according to the invention including the lactobacilli is in the form of a free-flowing particulate material or granule, which is capable to be pressed with high speed by a standard tablet machine directly into chewing gum tablets of different shapes, forms, and weights. Finally, the chewing gum is finished and packed in a conventional manner. The chewing gums typically contain an effective dose of lactobacilli per chewing gum, e.g., Lactobacillus reuteri DSM 17648, which may be between 1×10³ and 1×10¹³ cells in living, viable, dead, or fragmented forms as cell wall constituents. The preferred dose includes 1×10⁹ to 1×10¹¹ lactobacilli per application.

Further information about the composition of corresponding chewing gums can be taken from the literature, e.g., Biswal et al., International Journal of Advances in Pharmacy, Biology and Chemistry, Vol. 2(2), p.351ff.

The chewing gums according to the invention may contain further active ingredients, such as, e.g., emulsifiers, wetting agents, antibiotics, acid regulators, gastric acid blockers, or inflammation inhibitors. Preferably, however, they include only Lactobacillus cells, preferably DSM 17648, as the effective component.

It turned out that by regular application of such chewing gums and/or other preparations according to the invention, the H. pylori treatment is significantly improved. In particular, the colonisation of the upper airways and the oesophagus with H. pylori is reduced, furthermore, the recolonisation of the gastro-intestinal tract is significantly reduced.

Without intending to be bound to a certain theory, we assume that plaque and gingival pockets or biofilms existing in the upper airways and the oesophagus, in particular when chronic inflammations of the oral epithelia exist, e.g., in the case of chronic periodontal disease or chronically recurrent aphthae, may possibly contain H. pylori cells, which cannot sufficiently be fought by conventional treatment forms. By using the chewing gums according to the invention and/or other preparations according to the invention, these hidden centres of infection can also be fought in a better way.

The chewing gums according to the invention and/or other preparations according to the invention are typically administered over a period of time of six days to six months. The application may take place several times per day. Typically, four to six chewing gums approximately every two hours per day over a period of time of 28 days will result in an effective treatment. The application of such chewing gums may also occur prophylactically, e.g., when travelling in high-risk areas or in the case of contact with H. pylori-infected persons. The prophylactic application of such chewing gums is particularly advantageous since the application of classic antibiotics for the prevention is out of question due to the risk of developing resistance.

### EXAMPLES

### 1) Composition of the Gum Base

| | |
|---|---|
| Gum base | 22-26% |
| Xylitol | 8-12% |
| Plasticiser | max. 2% |
| Anticaking agent (E-551) | max. 2% |
| Antioxidant (BHT) | max. 260 ppm |
| Sorbitol | up to 100% |

### 2) Composition of the Chewing Gums According to the Invention

Lactobacillus cells, e.g., L. reuteri DSM 17648 50 mg/gum

| | |
|---|---|
| Gum base Hig PWD-01 | 90% |
| Gum (in gum base) | 21.6% |
| Sorbitol (in gum base) | 57.6% |
| Xylitol (in gum base) | 10.5% |
| Flavour (mint) | |
| Var. additives | ad 100% |

Typically, 50 mg L. reuteri include 5×10⁹ cells.

### 3) Application of the Chewing Gums According to the Invention

a) Probands with chronic periodontal disease and H. pylori colonisation in dental plaques of deep gingival pockets receive 4-6 chewing gums according to Example 2 per day, which are chewed preferably in the morning, at midday, in the afternoon, and the evening for 20-40 minutes each after the meals. After chewing four to six chewing gums, an average reduction of the H. pylori density in dental plaque samples and mouth rinsing samples by 20% was observed.
b) Probands with chronic periodontal disease and evidence of H. pylori in dental plaque of deep gingival pockets and simultaneously present dyspepsis receive 4 to 6 chewing gums according to Example 2 per day, which are chewed preferably in the morning, at midday, in the afternoon, and the evening for 20-40 minutes each after the meals. After 28 days, significantly less H. pylori in dental plaques is detected, clinical signs of periodontal disease have been significantly improved for more than half of the patients, dyspeptic complaints have been significantly improved for 80% of the patients.

### 4) Production of Buccal Films

a) The films are produced by the modified casting solvent vaporisation method (Angela Abruzzo et al., Pharmaceutics 2020, 12(3), 241). HPMC (2.5 wt. %) is dissolved in PG-containing water (1 wt. %), and the solution is stirred for 8 h until a viscous, gel-type solution is formed (viscosity 9100 mPa·s, pH 6.3). Optionally, biodegradable and simultaneously physiologically tolerated polymers for delaying the dissolution (e.g., polylactic acid, polyvinyl alcohol, xanthan gum) may be added to the solution. Then, the viscous solution is left for at least 8 hours at room temperature, in order to assure a clear, bubble-free gel. The bubble-free gel (6.7 g) was poured onto a Petri dish (5 cm diameter) and dried in the oven at 50° C. for 5 h. After this time, the dried Lactobacillus preparation is applied onto one side of the film (loaded side). Glass rings of suitable size (height, 1 cm; diameter, 1 cm) are used to include a sufficient amount (10 mg, corresponding to 9.21±0.25 log CFU) of Lactobacillus cells and to cut the film into the final administration forms. Ultimately, the Petri dish is brought for at least one day into an exsiccator at 20° C., in order to complete the film formation (up to constant weight).
   The further characterisation of the buccal film is performed according to Angela Abruzzo et al., Pharmaceutics 2020, 12(3), 241.
b) Probands with chronic periodontal disease and evidence of H. pylori in dental plaque of deep gingival pockets and simultaneously present dyspepsia receive 5 of the above buccal films, which are placed preferably in the morning, at midday, in the afternoon, and the evening respectively into the oral cavity (inner side of the cheek) and dissolved there over a period of time of 2-30 minutes.
   Here, too, significantly less H. pylori in dental plaques is detected after 28 days, clinical signs of periodontal disease have been significantly improved for more than half of the patients, dyspeptic complaints have been significantly improved for more than 75% of the patients.

1. A composition comprising a physiologically effective dose of Lactobacillus cells in the form of a) a chewing gum, b) another preparation with prolonged residence time in the mouth, or c) a preparation with application in the upper airways, for the treatment of H. pylori colonisation of the upper airways, the oesophagus, and/or the digestive system.

2. The composition according to claim 1, wherein the Lactobacillus cells are selected from the group Lactobacillus crispatus, Lactobacillus gasseri, Lactobacillus lactis, Lactobacillus helveticus, Lactobacillus acidophilus, Lactobacillus bulgaricus, Lactobacillus delbrueckii, Lactobacillus casei, Lactobacillus paracasei, Lactobacillus pentosus, Lactobacillus rhamnosus and Lactobacillus plantarum, Lactobacillus reuteri, Lactobacillus brevis, Lactobacillus fermentum, Lactobacillus viridescens and/or Bifidobacterium bifidum.

3. The composition according to claim 1, comprising an effective dose of Lactobacillus cells per application between 10³ and 10¹³ cells in living, viable, dead, or fragmented forms as cell wall constituents or isolated cell wall molecules respectively from 1×10³ to 1×10¹³ cells per application.

4. The composition according to claim 3, comprising an effective dose of Lactobacillus cells per application between 10⁹ and 10¹¹ cells in living, viable, dead, or fragmented forms as cell wall constituents or isolated cell wall molecules respectively from 10⁹ to 10¹¹ cells per application.

5. The composition according to claim 1, comprising Lactobacillus reuteri as filed under DSM 17648 in the form of a chewing gum for the treatment of H. pylori colonisation of the oral cavity, the upper airways, the oesophagus, the stomach, and the intestine.

6. The composition according to claim 1, comprising Lactobacillus cells in viable or dead form.

7. The composition according to claim 1 in an administration form, selected from
buccal tablets (also muco-adhesive buccal tablets) or orodispersible tablets,
oral active ingredient films,
gargle solutions or concentrates (incl. powders/tablets for the production),
mouth rinses or concentrates (incl. powders/tablets for the production), gingival cleaning solutions,
oromucosal preparations, such as drops, gel, liquids, pastes, solutions, sprays, and/or suspensions,
sublingual preparations, such as sprays, tablets, films,
lozenges or candies,
powders or granules for oral application,
preparations for nasal application, such as nose drops, liquid nose sprays, nose powders, semi-solid preparations for nasal application, nose rinses, or nose inhalers.
